(19) United States
(12) Patent Application Publication
(10) Pub. No.: US 2010/0129853 A1
(43) Pub. Date: May 27, 2010

(54) DISEASE MARKERS

(75) Inventors: David Wraith, Bristol (GB); Heather Streeter, Bristol (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Apitope Technology (Bristol) Limited, Bristol (GB)

(21) Appl. No.: 12/597,416

(22) PCT Filed: Apr. 23, 2008
(86) PCT No.: PCT/GB2008/001415

§ 371 Date: Jan. 8, 2010

(30) Foreign Application Priority Data
Apr. 24, 2007 (GB) 0707933.8

(51) Int. Cl. C12Q 1/02; G01N 33/68; G01N 33/48; G01N 33/53

(52) U.S. Cl. 435/29; 436/86; 436/94; 436/501

(57) ABSTRACT

The present invention relates to biomarkers for autoimmune disease, and in particular to a method for determining a status of an autoimmune disease in a test subject, comprising measuring production of IL-5 and IL-13, as well as to associated uses and kits.

The present invention relates to the field of methods for evaluating autoimmune disease in a subject, in particular for detecting or determining a risk of developing a disease such as multiple sclerosis.

The immune system is a primary defence mechanism against diseases caused by pathogens such as bacteria and viruses, as well as against diseases caused by abnormal growth of body tissues (such as in cancer). The immune system is typically able to distinguish between normal body cells (designated “self”) and foreign pathogens or abnormal cells (”non-self”). Tolerance is a mechanism which leads the immune system to avoid reacting against self antigens. However in autoimmune disease, the immune system loses the ability to recognize “self” as normal, and this leads to an immune response directed against body tissues or cells. The pathologies resulting from autoimmunity often have serious clinical consequences and are a major health problem which is currently inadequately addressed.

One example of an autoimmune disorder is multiple sclerosis (MS), a progressive disease of the central nervous system (CNS). In MS patients, the patient's immune system destroys myelin, the protective layer that surrounds and insulates the nerve fibers in the brain and spinal cord. The destruction of the myelin sheath leads to disruption of neurotransmission and scarring damage to the nerve fibres. The end result is the manifestation of numerous symptoms in the affected patient including tingling or numbness, slurred speech, and impaired vision. Over the course of the disease, there is loss of strength in the extremities, leading to problems with movement and in the most severe cases, leading to paralysis of the limbs. Based on clinical diagnosis, there are currently four types of MS classifications, based on which part of the brain or spinal cord are affected, severity and frequency of attacks.

Initial attacks of MS are often transient or mild, which may mean that medical intervention is not sought at an early stage. The most common initial symptoms reported are changes in sensation or partial vision loss (optic neuritis). Multiple sclerosis is often difficult to diagnose in its early stages, since at least two separate episodes of characteristic neurological symptoms are required to confirm that a patient is suffering from the disease. Magnetic resonance imaging (MRI) also may be used in diagnosis to identify areas of demyelination, and testing of cerebrospinal fluid (CSF) can provide evidence of chronic inflammation of the central nervous system. Measurement of antibodies against myelin proteins such as myelin oligodendrocyte glycoprotein (MOG) and myelin basic protein (MBP) has also been suggested as being useful as a diagnostic tool in MS. However, all of these methods have limitations and may not be practical for analysing large numbers of subjects, including those who are apparently healthy or are in the early stages of the disease.

There is therefore a need for improved methods of detecting and diagnosing autoimmune diseases such as multiple sclerosis. In particular, there is a need for methods which may allow early detection of the disease, or provide an indication of disease susceptibility in individuals who may be asymptomatic.

Accordingly, the present invention provides in one embodiment a method for determining a status of an autoimmune disease in a test subject, comprising measuring production of a Th2 cytokine by leukocytes from the subject.

In another embodiment, the invention provides use of a Th2 cytokine as a biomarker for determining a status of an autoimmune disease in a test subject.

In another embodiment, the invention provides a kit for determining status of an autoimmune disease in a test subject, comprising one or more reagents suitable for measuring production of a Th2 cytokine by leukocytes from the test subject.

By “determining a status of an autoimmune disease” it is intended to include any method which involves evaluating whether the disease is present in the test subject, whether the disease may develop in the test subject in the future, and/or the severity or form of any present or future disease. Thus the method may involve any diagnostic or prognostic method, for instance a method of detecting the disease, a method of diagnosing the disease, a method of monitoring disease progression, a method of evaluating disease prognosis, a method of predicting disease outcome, a method of determining a risk of developing the disease or a method of predicting susceptibility to the disease.

Examples of the autoimmune diseases include, but are not limited to: rheumatoid arthritis (RA), myasthenia gravis (MG), multiple sclerosis (MS), systemic lupus erythematosus (SLE), autoimmune thyroiditis (Hashimoto's thyroiditis), Graves' disease, inflammatory bowel disease, autoimmune uveoretinitis, polymyositis and certain types of diabetes.

Preferably the autoimmune disease is a demyelinating disorder, more preferably a CNS demyelinating disorder. Examples of non-CNS demyelinating disorders include acute demyelinating polyneuropathy (Guillain Barre syndrome) and chronic inflammatory demyelinating neuropathy. Examples of CNS demyelinating disorders include MS, progressive multifocal leukoencephalopathy (PML), acute disseminated encephalomyelitis (ADEM), Device syndrome and Balo disease. The disease may also be a secondary demyelinating disorder such as CNS lupus erythematodes, Sjogren's syndrome, or isolated cerebral vasculitis.

Most preferably the autoimmune disease is multiple sclerosis. The method may be used to evaluate any form or subtype of MS, including relapsing-remitting (RR), secondary progressive (SP), primary progressive (PP) and progressive relapsing (PR) multiple sclerosis.

The test subject is preferably a mammal, more preferably a human. The test subject may be, for example, an individual who is suspected to be suffering from the autoimmune disease (for instance due to the presence of other symptoms of the disease) and it is desired to confirm or rule out presence of the disease. Alternatively, the test subject may be an individual who has already been diagnosed as suffering from the disease, for instance in embodiments where the method is performed to monitor disease progression or to determine the severity of the disease in the subject. In another embodiment, the subject may be an apparently healthy (asymptomatic) individual, for instance where it is desired to screen for early stage disease, or for susceptibility to developing the disease in the future.

The method of the present invention involves measuring production of a Th2 cytokine. By “Th2 cytokine” it is meant a cytokine which is associated with a Type 2 helper T cell (Th2 ) response. In one embodiment, the Th2 cytokine is encoded by a gene located on human chromosome 5 (or mouse chromosome 11), in particular a gene which is part of the cytokine gene cluster on human chromosome 5q23-31. Preferably the Th2 cytokine is IL-4 (interleukin 4), IL-5 (interleukin 5), or IL-13 (interleukin 13). In one embodiment, expression of the Th2 cytokine is regulated by a GATA-3 transcription factor. The invention also encompasses methods in which production of two or more Th2 cytokines in a subject is measured, including any combination of those cytokines mentioned above (e.g. IL-4 and IL-5, or IL-4, IL-5 and IL-13).

The Th2 cytokine may be derived from any species corresponding to the test subject. The method preferably involves measuring production of human Th2 cytokines, e.g. human IL-4, human IL-5 or human IL-13.

By “measuring production of a Th2 cytokine” it is intended to include any method which directly or indirectly enables levels of the Th2 cytokine (e.g. IL-5) to be determined. Thus the method encompasses detecting a derivative or fragment of the Th2 cytokine as well as the native full-length protein or mRNA sequence. Derivatives and fragments include variants which arise as a result of a deletion, substitution or insertion of one or more amino acid or nucleotide residues, for instance as a result of naturally occurring gene variability. Derivatives may also arise as a result of the processing of the gene or gene product within the body and/or a degradation product. Modifications at the protein level can be due to enzymatic or chemical modification within the body. For example the modification can be a glycosylation, phosphorylation or farnesylation.

Th2 cytokine production in the subject may be measured by any suitable method, including determining cytokine mRNA and/or protein production in the test subject. For example, methods for detecting IL-5 protein may include the use of an antibody, capture molecule, receptor, or fragment thereof which binds to IL-5. Antibodies which bind to IL-5 and other Th2 cytokines are known (see for example Abrams et al., “Strategies of anti-cytokine monoclonal antibody development: immunoassay of IL-10 and IL-5 in clinical samples.” Immunol Rev 127: 5-24; Schumacher, et al., “The characterization of four monoclonal antibodies specific for mouse IL-5 and development of mouse and human IL-5 enzyme-linked immunosorbent.” J Immunol 141(5): 1576-81) or may be produced by methods known in the art, including immunization of an animal and collection of serum (to produce polyclonal antibodies) or spleen cells (to produce hybridomas by fusion with immortalised cell lines leading to monoclonal antibodies). Methods for detecting IL-5 mRNA may include the use of a complementary nucleic acid probe, for example a single-stranded DNA probe, or real-time PCR. Detection molecules such as antibodies or nucleic acid probes may optionally be bound to a solid support such as, for example, a plastic surface or beads or in an array. Suitable test formats for detecting IL-5 protein include, but are not limited to, an immunoassay such as an enzyme-linked immunosorbent assay (ELISA), radioimmunoassay (RIA), Western blotting and immunoprecipitation. Suitable mRNA detection methods include Northern blotting and reverse transcriptase polymerase chain reaction (RT-PCR). Other Th2 cytokines, such as IL-4 and IL-13, may be detected using analogous methods and reagents to those discussed above in relation to IL-5, but which are specific for those other cytokines.

Alternatively the level of Th2 cytokine production may be determined by mass spectroscopy. Mass spectroscopy allows detection and quantification of a cytokine protein by virtue of its molecular weight. Any suitable ionization method in the field of mass spectroscopy known in the art can be employed, including but not limited to electron impact (EI), chemical ionization (CI), field ionization (FDI), electrospray ionization (ESI), laser desorption ionization (LDI), matrix assisted laser desorption ionization (MALDI) and surface enhanced laser desorption ionization (SELDI). Any suitable mass spectrometry detection method may be employed, for example quadrapole mass spectroscopy (QMS), fourier transform mass spectroscopy (FT-MS) and time-of-flight mass spectroscopy (TOF-MS).

In one embodiment, Th2 protein secretion is determined by cytometric bead array (CBA). The CBA (or “multiplexed bead assay”) consists of a mixture of different types of bead, which are uniform in size but which are distinguishable by virtue of different fluorescence intensities of an emitting dye. A capture molecule such as an antibody is covalently coupled to a particular type of bead, and the analyte (e.g. IL-5) bound to the antibody is detected by a fluorescence-based emission and flow cytometric analysis. In an assay of this type, multiple Th2 cytokines may be detected by coupling antibodies specific for each cytokine (e.g. IL-4, IL-5 and IL-13) to different types of bead.

The method of the invention involves measuring Th2 cytokine production by leukocytes from the subject. Preferably the method is performed in vitro, i.e. Th2 cytokine production is determined in an isolated sample (comprising leukocytes) derived or extracted from the test subject. The sample may be a solid tissue sample or may be derived from a body fluid, such as, but not limited to, lymph, urine, blood, serum, plasma, fecal matter, cerebrospinal fluid or saliva. Preferably the sample is derived from blood or lymph. More preferably the sample is enriched in, or comprises a purified population of leukocytes, particularly lymphocytes and/or monocytes. For instance, in one embodiment the sample comprises peripheral blood mononuclear cells extracted from the test subject.

Preferably the level of the Th2 cytokine in the isolated sample is compared with a control level. The control level of the Th2 cytokine may be determined, for example in a control sample derived from a healthy subject. The control sample may be derived from the same tissue type as the sample derived from the test subject. A reduced level of the Th2 cytokine, e.g. IL-4, IL-5 or IL-13, in the isolated sample from the test subject (compared to the level in the control sample) is typically indicative of the autoimmune disease in the test subject, e.g. indicates that the subject has the disease, or may be likely to develop the disease in the future. In some embodiments, a quantitative analysis of the relative level of Th2 cytokine production in the test and control subjects may allow an evaluation of disease prognosis, e.g. severity or likely speed of progression. For instance, a greater reduction in IL-5 (or IL-4 or IL-13) production relative to control may be indicative of a worse prognosis.

In preferred embodiments, the isolated sample is challenged with an agent that typically stimulates production of the Th2 cytokine. The agent preferably stimulates production of the Th2 cytokine in a control sample from a healthy subject. For example, IL-5 production in the isolated sample may be determined in response to the challenge, and compared to IL-5 production in the control sample following the same challenge. In one embodiment, the agent is purified protein derivative of Mycobacterium tuberculosis.

Preferably the agent is a T cell activator. Any agent which activates T cells in an antigen-dependent or antigen-independent manner may be used. The agent may activate T cells by binding to a T cell receptor or via any other mechanism. Suitable T cell activating agents include phytohaemagglutinin (PHA, a lectin that stimulates all human T cells); anti-CD3 or anti-CD28 antibodies, optionally coated on beads; phorbol 12-myristate 13-acetate (PMA, phorbol ester); and ionomycin (a calcium ionophore). Combinations of the above agents may also be used, e.g. PHA and PMA.

In another embodiment the T cell activator is Cytostim. CytoStim was developed for rapid and efficient restimulation of human effector/memory T cells. CytoStim is an antibody-based reagent that acts similar to a superantigen but independently of certain Vβ domains of the TCR. It causes activation of T cells by binding the T cell receptor (TCR) and crosslinking it to an MHC molecule of an antigen-presenting cell (APC). Upon stimulation with CytoStim, CD4+ and CD8+ T cells start to secrete cytokines or up-regulate activation markers on their cell surface within a few hours.

In another embodiment, the isolated sample is challenged with an autoantigen, which may be an autoantigen against which an immune response is directed in the autoimmune disease which it is desired to detect. Preferably the agent comprises myelin basic protein (an autoantigen associated with MS and other demyelinating disorders) or a fragment thereof (for example in an embodiment where the autoimmune disease is a demyelinating disorder such as MS).

In one embodiment, presence or susceptibility to the disease is indicated if the level of Th2 cytokine (e.g. IL-5, IL-4 or IL-13) production is reduced by at least 25%, at least 50%, at least 75%, or at least 90% with respect to a control level. The level of the cytokine may be compared at any suitable time point after challenge with the agent, preferably 6 hours to 28 days, more preferably 12 hours to 14 days, most preferably 1 to 10 days after the challenge, for example, 24 hours, 4 days or 8 days after the challenge.

In another embodiment, the invention provides a kit for performing a method as described herein, for example a kit for measuring production of a Th2 cytokine by leukocytes from a test subject (e.g. in a sample comprising leukocytes derived from the test subject). The kit may thus be suitable for screening, diagnosis, and/or evaluating prognosis of an autoimmune disease, such as MS. The kit may comprise one or more reagents which specifically bind to a Th2 cytokine protein or mRNA (for instance an antibody which binds to the Th2 cytokine and which allows detection and quantification of the cytokine protein), and optionally one or more buffers, detectable labels, or other reagents as may be useful in a particular assay. In one embodiment, the kit further comprises an agent which typically stimulates production of a Th2 cytokine, for instance in a control sample from a healthy subject. Thus the agent may be a T cell activator or an autoantigen which is associated with the autoimmune disease to be tested for. The kit may further comprise instructions for performing the methods described herein, for example instructions for using such reagents to determine a status of an autoimmune disease in a test subject, e.g. for determining whether the subject has the disease or is susceptible to the disease.

The method will now be described by way of example only with reference to the following specific embodiments, in which:

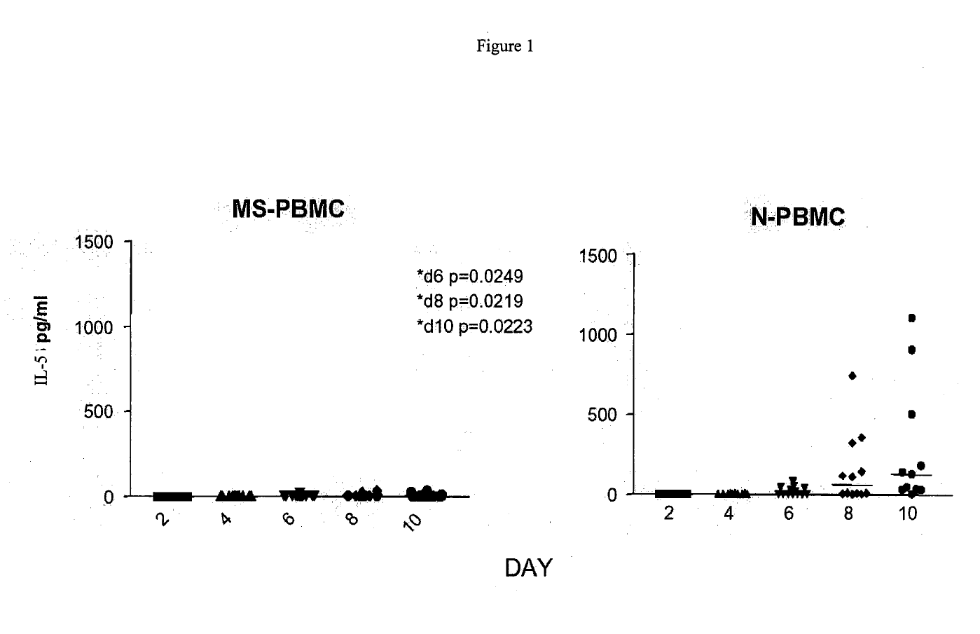

FIG. 1 shows IL-5 secretion by peripheral blood mononuclear cells from healthy subjects and MS patients, at various times after treatment with myelin basic protein;

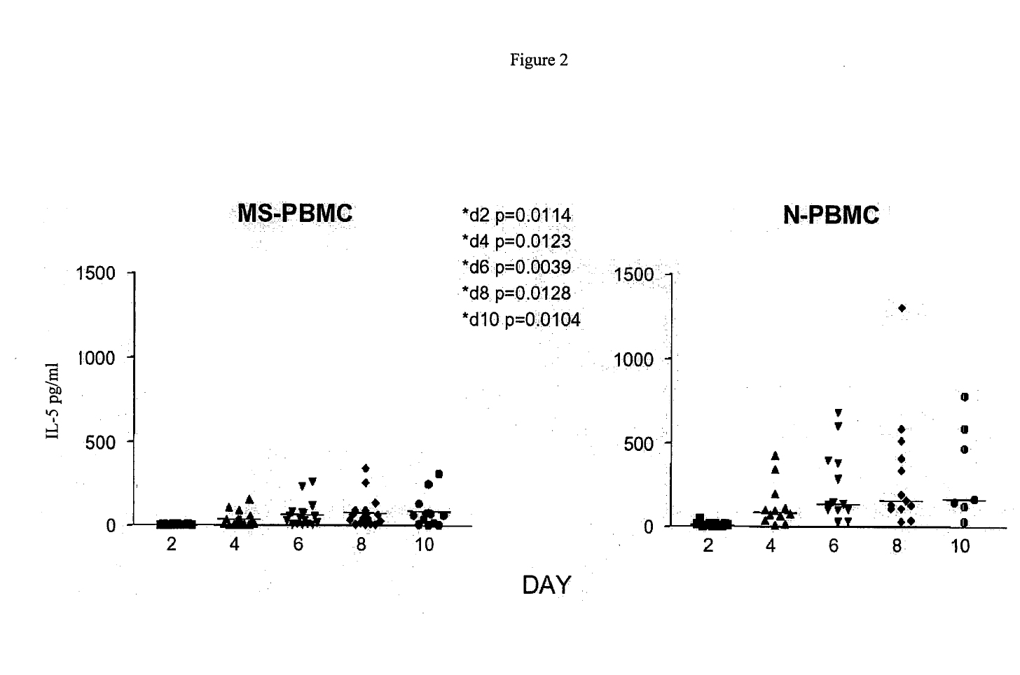

FIG. 2 shows IL-5 secretion by peripheral blood mononuclear cells from healthy subjects and MS patients, at various times after treatment with purified protein derivative of Mycobacterium tuberculosis;

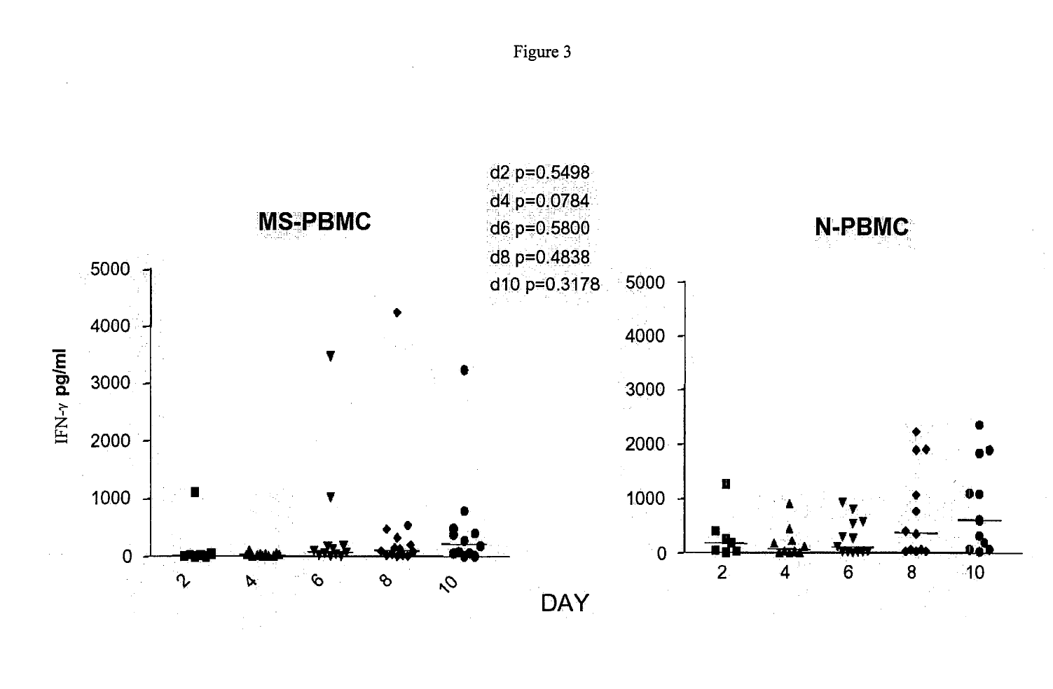

FIG. 3 shows IFN-γ secretion by peripheral blood mononuclear cells from healthy subjects and MS patients, at various times after treatment with myelin basic protein;

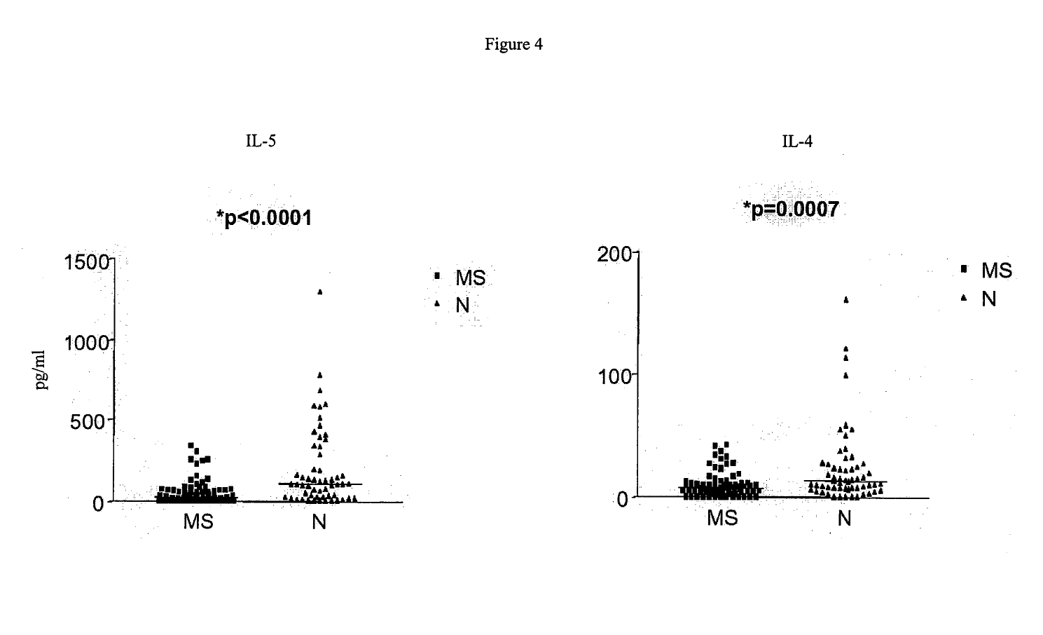

FIG. 4 shows IL-5 and IL-4 secretion by peripheral blood mononuclear cells from healthy subjects and MS patients in response to myelin basic protein, using data from all time points.

The differentiation of T helper (Th) lymphocytes plays a crucial role in mounting an effective immune response and in establishing immunological memory to a pathogen. Naïve Th cells stimulated by antigen and polarizing signals can develop into effector cells with distinct cytokine profiles: Th1 cells express IFN-γ and thereby activate cell-mediated immune responses, whereas Th2 cells activate B cell proliferation and antibody production by expression of IL-4, IL-5, and IL-13. Polarizing signals that induce Th1 and Th2 differentiation are IL-12 and IL-4, respectively.

According to embodiments of the present invention, production of Th2 cytokines by lymphocytes is linked to disease status in subjects suffering from an autoimmune disease such as MS. In contrast, production of Th1 cytokines such as IFN-γ is not related to disease state. A number of Th2 cytokines of interest in the present invention are discussed below in more detail.

Human IL-5 (UniProtKB/Swiss-Prot accession no. P05113) is a glycoprotein comprising a homodimer of two polypeptide chains, each chain comprising 115 amino acid residues (Tanabe et al., “Molecular cloning and structure of the human interleukin-5 gene.”, J. Biol. Chem. 262:16580-16584(1987); Milburn et al., “A novel dimer configuration revealed by the crystal structure at 2.4A resolution of human interleukin-5”, Nature 1993; 363(6425):172-176.). IL-5 is typically produced by T cells and mast cells, and may function to stimulate B cell growth, to increase immunoglobulin secretion, and to induce eosinophil activation.

IL-4 is a Th2 cytokine involved in the stimulation of activated B-cell and T-cell proliferation, and the differentiation of CD4+ T-cells into Th2 cells. Human IL-4 (UniProtKB/Swiss-Prot accession no. P05112) is produced from a 153 amino-acid precursor which is cleaved to produce a 129 amino acid glycoprotein in its mature form (Yokota et al., “Isolation and characterization of a human interleukin cDNA clone, homologous to mouse B-cell stimulatory factor 1, that expresses B-cell- and T-cell-stimulating activities.”, Proc. Natl. Acad. Sci. U.S.A. 83:5894-5898(1986)).

IL-13 is closely related to IL-4, and both of these cytokines have similar activities on immune cells in inflammatory and allergic conditions. The receptor for IL-13 is a multi-subunit receptor that includes the alpha chain of the IL-4 receptor (IL-4Rα), and at least one of two known IL-13-specific binding chains. The transcription factor, signal transducer and activator of transcription 6 (STAT6) is linked to the regulation of expression of both IL-4 and IL-13. Human IL-13 (UniProtKB/Swiss-Prot accession no. P35225) is a 112 amino acid glycoprotein formed from a precursor by the removal of a 20 residue signal sequence (Minty et al., “Interleukin-13 is a new human lymphokine regulating inflammatory and immune responses.”, Nature 362:248-250(1993); McKenzie et al., “Interleukin 13, a T-cell-derived cytokine that regulates human monocyte and B-cell function.”, Proc. Natl. Acad. Sci. U.S.A. 90:3735-3739(1993); Dolganov et al., “Coexpression of the interleukin-13 and interleukin-4 genes correlates with their physical linkage in the cytokine gene cluster on human chromosome 5q23-31.” Blood 87:3316-3326(1996)).

The genes for IL-3, IL-4, IL-5, IL-9, IL-13 and granulocyte-macrophage colony-stimulating factor (GM-CSF) are contained within a tightly linked cluster on human chromosome 5 and mouse chromosome 11 (Lee et al., “The IL-4 and IL-5 genes are closely linked and are part of a cytokine gene cluster on mouse chromosome 11” Somat Cell Mol Genet 1989; 15(2):143-152; van Leeuwen et al., “Molecular organization of the cytokine gene cluster, involving the human IL-3, IL-4, IL-5, and GM-CSF genes, on human chromosome 5”, Blood 1989; 73(5):1142-1148). These genes are often co-expressed in helper T cells in a Th2 type response.

Various transcription factors are involved in the regulation of expression of Th2 cytokines. GATA 3 (GATA binding protein 3) controls the production of a range of cytokines including IL-4, IL-5 and IL-13 (Stevens et al. Eur. J. Immunol. 36, 3305. 2006). Histone deacetylase 4 and p300 (known as histone acetyltransferase) control the activity of the IL-5 promoter. C/EBP beta, GATA 3, NFAT and YY1 are transcription factors binding to the IL-5 promoter and are essential for recruitment of histone deacetylase 4 (Han et al. Biochemical Journal 400, 439. 2006).

AP-1 and GATA-3 are known to bind to the IL-5 promoter. Mutagenesis has shown that the Ets/NFAT site is of critical importance along with AP-1 and GATA-3 in regulating IL-5 (Wang et al. International Immunology 18, 313. 2006). Bcl-3 regulates differentiation of cells secreting IL-4, IL-5 and IL-13. Bcl-3 deficient cells show impaired production of all three cytokines and these cells exhibit decreased GATA-3 (Corn et al. Journal of Immunology 175, 2102. 2005)

Forced expression of GATA-3 in T cells results in the induction of Th2 cytokines IL-4 and IL-5 (Sundrud et al. Journal of Immunology 171, 3542. 2003). Bcl-6 inhibits GATA-3 expression at the post transcriptional level (Kusam et al. Journal of Immunology 170, 2435. 2003)

GATA-3 has been found to be pivotal for Th2 cytokine memory by inducing Th2 cytokine expression and inhibiting IFN-γ. In naive Th cells, GATA-3 occurs in low concentration. A slow and long-lasting up-regulation of its expression is induced by Th2 -polarizing conditions, simultaneous IL-4 delivery and T cell receptor (TCR) stimulation by antigen. GATA-3 transcription is activated by Stat6, which itself is under the control of the IL-4 signal. Thus expression of GATA-3 is important in the control of Th2 cytokine production. There is divergence in the role of GATA-3 in that the N-terminal finger of the protein influences IL-5 while the C-terminal finger controls IL-4 and IL-13.

Thus, without being bound by theory, reduced production of Th2 cytokines (such as IL-4, IL-5 and IL-13) by lymphocytes from subjects suffering from an autoimmune disease such as MS may be linked to differences in gene regulation by transcription factors such as GATA-3.

### Examples

Abbreviations

PBMC, peripheral blood mononuclear cells; PPD, purified protein derivative of Mycobacterium tuberculosis; MBP, myelin basic protein; CBA, cytometric bead array; qRT-PCR, quantitative real-time polymerase chain reaction.

Collection and Storage of PBMC in Liquid Nitrogen

Citrated peripheral blood samples were collected from healthy individuals from whom written informed consent had been obtained, and from MS patients. PBMC isolated by density centrifugation on Histopaque-1077 (Sigma, Poole, UK) as previously described, were either set up immediately in culture (fresh PBMC) or cryopreserved in liquid nitrogen (frozen PBMC) for culturing at a later date.

For freezing PBMC, aliquots of 20×10⁶ cells ml⁻¹ in 40% a MEM (Gibco Invitrogen Ltd; Paisley, UK) were supplemented with 20 Mm HEPES, 100 Uml⁻¹ penicillin, 100 μgm1⁻¹ streptomycin sulphate, 4 mM glutamine (all from Sigma, Poole, UK), 50% heat inactivated autologous plasma and 10% Dimethyl Sulphoxide, all at 25° C. Cell aliquots were then transferred to a Nalgene Cryo 1° C. freezing container (Nalge Europe Ltd; Hereford, UK), and stored at −70° C. overnight before transferring to liquid nitrogen the next day. When required for culture, cell aliquots were thawed rapidly, washed twice in tissue culture medium containing 5% heat inactivated, autologous plasma and resuspended at the appropriate concentration for culture. Viability of all cell preparations, as estimated by Trypan blue exclusion, was in excess of 95%.

Cell Culture

Fresh or cryopreserved PBMC were set up in 1 ml cultures containing 1.5×10⁶ cells in α-MEM in 48 well tissue culture plates (Nunc International, Costar, Corning Inc. New York USA). Responses to PPD (M.A.A.F: Central Vet. Laboratory, Addlestone, Surrey, UK) and human MBP both at 50 μgml⁻¹ were monitored over a period of 10 days. Control wells contained no antigen. After 20 hrs or 2, 4, 6, 8 and 10 days of culture, duplicate 100 μl aliquots of cell suspension were removed from each 1 ml culture to measure proliferation in response to antigen by uptake of [³H] thymidine. Background [³H] thymidine uptake observed in cultures containing no antigen was subtracted from the corresponding PPD or MBP response and results were expressed as δccpm.

The remaining cell suspension was centrifuged and RNA extracted from the cell pellet. The supernatant was collected, aliquoted and stored at −20° C. The RNA and supernatant samples were used to measure cytokine (IL-4, IL-5 and IFN-γ) gene induction and secretion respectively in PBMC at intervals during the culture period.

Measurement of Secreted Cytokines: Cytometric Bead Array Assay

Culture supernatant cytokine levels were determined using the Cytometric Bead Assay (BD™ CBA Human Th1/Th2 cytokine kit, Becton Dickenson Biosciences, Cowley, Oxford, UK) following the manufacturer's instructions. Following acquisition of sample data using the FACS Calibur (BD Biosciences), results were generated in graphical and tabular form using BD CBA software. The minimum quantifiable levels of cytokines were: IL-5 2.4 pgml⁻¹, IL-4 2.6 pgml⁻¹ and IFN-γ 7.1 pgml⁻¹.

Cytokine Secretion in Response to Challenge with MBP or PPD

As shown in FIGS. 1 and 2, treating PBMCs derived from healthy subjects with MBP or PPD results in IL-5 secretion between 4 and 10 days after treatment. IL-5 secretion in response to MBP or PPD is significantly reduced in PBMC preparations derived from MS patients. However, IFN-γ secretion in response to MBP does not significantly differ between PBMCs from healthy subjects and MS patients, as shown in FIG. 3. An analysis of data from all time points studied shows that production of both IL-4 and IL-5 is reduced in PBMCs from MS patients (see FIG. 4).

### Further Examples

In further examples, PBMCs are obtained from individuals including apparently healthy subjects, subjects showing one or more clinical symptoms which may be indicative of early stage MS, or subjects with a confirmed diagnosis of MS. IL-4, IL-5 and IL-13 secretion by PBMCs from each individual is determined in response to PPD and/or MBP challenge using the methods described above. For each individual, Th2 cytokine secretion is compared to a control level based on a mean value for secretion from a plurality of healthy subjects. In apparently healthy individuals, a reduced level of IL-4, IL-5 and/or IL-13 secretion relative to control may indicate a predisposition to develop MS, or early stage disease, suggesting that further investigations are made to confirm the diagnosis and/or that prophylactic treatment may be indicated. In subjects already showing characteristic clinical symptoms, a reduced level of Th2 cytokine secretion may confirm a diagnosis of MS and indicate initiation of treatment, for instance based on interferon-beta. In subjects with a confirmed diagnosis of MS, the relative reduction in Th2 cytokine secretion compared to control may be used to monitor disease progress.

1-31. (canceled)

32. A method for determining a status of an autoimmune disease in a test subject, comprising measuring production of IL-5 and IL-13.

33. The method according to claim 32, wherein the disease is a demyelinating disorder.

34. The method according to claim 33, wherein the disease is multiple sclerosis.

35. The method of claim 32, wherein the method further comprises providing an isolated sample comprising leukocytes derived from the test subject; and determining a level of L-5 and IL-13 in the isolated sample.

36. The method according to claim 35, further comprising comparing the determined level of IL-5 and IL-13 in the isolated sample with a control level of IL-5 and IL-13.

37. The method according to claim 36, wherein the control level of IL-5 and IL-13 is determined in a control sample derived from a healthy subject, and a reduced level of IL-5 and IL-13 in the isolated sample from the test subject is indicative of the autoimmune disease in the test subject.

38. The method according to claim 37, wherein production of IL-5 and IL-13 in the isolated sample from the test subject is determined in response to treating the isolated sample with an agent which stimulates production of IL-5 and IL-13 in a control sample from a healthy subject.

39. The method according to claim 38, wherein the agent comprises purified protein derivative of Mycobacterium tuberculosis.

40. The method according to claim 38, wherein the agent is a T cell activator.

41. The method according to claim 40, wherein the T cell activator is phytohaemagglutinin, an anti-CD3 antibody, an anti-CD28 antibody, phorbol 12-myristate 13-acetate, Cytostim or ionomycin.

42. The method according to claim 40, wherein the agent is an autoantigen associated with the autoimmune disease.

43. The method according to claim 42, wherein the agent comprises myelin basic protein or a fragment thereof.

44. The method according to claim 35, wherein the isolated sample is derived from blood or lymphatic tissue.

45. The method according to claim 44, wherein the isolated sample comprises a purified blood leukocyte preparation.

46. The method according to claim 32, wherein production of IL-5 and IL-13 is measured by determining the levels of IL-5 and IL-13 mRNA.

47. The method according to claim 32, wherein production of IL-5 and IL-13 is measured by determining the levels of secretion of IL-5 and IL-13.

48. The method according to claim 47, wherein secretion of IL-5 and IL-13 is determined by cytometric bead assay.

49. The method according to claim 32, wherein the test subject is human.

50. The method according to of claim 32, wherein determining status of the autoimmune disease in the test subject further comprises detecting the disease, diagnosing the disease, monitoring disease progression, evaluating disease prognosis, or predicting susceptibility to the disease.

51. A kit for determining status of an autoimmune disease in a test subject, comprising one or more reagents suitable for measuring production of IL-5 and IL-13 by leukocytes from the test subject.

52. The kit according to claim 51, further comprising an agent which promotes production of IL-5 and IL-13.

53. The kit according to claim 52, wherein the agent comprises a T cell activator.

54. The kit according to claim 52, wherein the agent is an autoantigen associated with the autoimmune disease.

55. The kit according to claim 54, wherein the autoimmune disease is multiple sclerosis and the agent is myelin basic protein.

56. The kit according to claim 52, wherein the kit further comprises antibodies which bind IL-5 and IL-13.
